(19) United States
(12) Patent Application Publication
(10) Pub. No.: US 2025/0065090 A1
(43) Pub. Date: Feb. 27, 2025

(54) DEVICE AND METHOD FOR DELIVERY OF A HEARING DRUG FORMULATION

(71) Applicant: Corti Therapeutics Pte. Ltd., Singapore (SG)

(72) Inventor: Qi-Ying HU, Needham, MA (US)

(73) Assignee: Corti Therapeutics Pte. Ltd., Singapore (SG)

(21) Appl. No.: 18/453,618

(22) Filed: Aug. 22, 2023

(51) Int. Cl. A61M 31/00; A61K 33/243

(52) U.S. Cl. CPC A61M 31/002; A61K 33/243; A61M 2210/0668

(57) ABSTRACT

A method of administering, to a tympanic cavity, a pharmaceutical composition using a drug delivery device implanted through a tympanic membrane, the method including fluidly connecting the drug delivery device to an injection system via an inlet of a flexible catheter of the drug delivery device; and introducing, to the tympanic cavity, a volume of the pharmaceutical composition via the drug delivery device.

### BACKGROUND

Cisplatin, also known as CDDP, is used extensively in the treatment of a broad range of neoplasms, including head and neck cancer, ovarian cancer, testicular cancer, and bladder carcinoma. Several other platinum based anticancer drugs have been developed and become a major component of cancer therapy; about half of all patients undergoing chemotherapy receive a platinum drug. However, the use of cisplatin and other platinum drugs are associated with side effects.

Ototoxicity is one of the dose limiting side effects. It is estimated that 60%-80% patients receiving cisplatin will develop hearing loss, which is irreversible. Notably, the cure rate with a cisplatin containing treatment is consistently increasing. For example, cisplatin based chemotherapy results in >90% cure rate for testicular cancer. Furthermore, cisplatin in combination with immune checkpoint inhibitors, such as programmed cell death 1 and ligand (PD-1/L1) has been shown to synergize with PD-1/L1 for antitumor activity. However, a majority of survivors developed irreversible hearing loss and other hearing disorders, such as tinnitus. Ototoxicity has also been frequently reported in patients receiving other platinum based anticancer drugs. This compromised the quality-of-life post-treatment and created a significant health and social burden. The hearing loss is irreversible, and there are no treatments or preventative approaches available as of today.

Both the therapeutic effects and the ototoxicity of CDDP are dose-dependent, and there is great interest in developing effective strategies to protect or rescue the auditory organ from CDDP ototoxicity without affecting the antitumoral activity of CDDP. While the exact mechanism for the ototoxicity induced by cisplatin or other platinum drugs is not entirely clear, it is reasonably believed that a portion of systemically dosed platinum enters the inner ear and causes cochlear damages in a dose-dependent manner.

There are some approaches to prevent platinum induced hearing loss, including platinum chelators (e.g., sodium thiosulfate (STS)) to inactivate platinum agents, dexamethasone to mitigate inflammation related damages, and N-acetylcysteine (NAC) to eliminate reactive oxygen species (ROS). NAC is also considered an efficient chelator. Among all strategies, chelators are the most promising.

Platinum (II) or platinum (IV) are considered a soft metal ion, therefore soft and/or intermediate soft chelating/coordinating groups should effectively inactivate platinum (II or IV) thiosulfate is an endogenous ion and sodium thiosulfate (STS) has also been used in clinic. At high concentration, it can efficiently bind to cisplatin and deactivate cisplatin.

Some systemic administration of STS has been evaluated in humans. It was found that STS can mitigate cisplatin induced hearing loss. However, it can potentially compromise the antitumor activity of cisplatin. Studies showed that STS can be administrated 6 hours post the end of infusion of cisplatin to potentially avoid interference of the antitumor activity. Furthermore, only specific non-disseminated cancer types are suitable to the preventative treatment without the potential compromise of the antitumor activity, and the protection is moderate.

In view of the potential for systemic STS to reduce the tumoricidal effects of platinum agents, an alternate mode of delivery is desired. As such, locally delivered sodium thiosulfate can be an attractive way to avoid such interference.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present disclosure.

### SUMMARY

The present disclosure relates to a method of administering, to a tympanic cavity, a pharmaceutical composition using a drug delivery device implanted through a tympanic membrane, the method including fluidly connecting the drug delivery device to an injection system via an inlet of a flexible catheter of the drug delivery device; and introducing, to the tympanic cavity, a volume of the pharmaceutical composition via the drug delivery device.

The present disclosure additionally relates to a drug delivery implant device, including a ventilation tube including a first end, a second end, and an opening extending from the first end to the second end; and a catheter including an inlet end and an outlet tip configured to be inserted through the ventilation tube via the opening, wherein an outer diameter of the catheter is less than an inner diameter of the opening of the ventilation tube, and the catheter is secured to the ventilation tube allowing for airflow through the opening of the ventilation tube.

Note that this summary section does not specify every embodiment and/or incrementally novel aspect of the present disclosure or claimed invention. Instead, this summary only provides a preliminary discussion of different embodiments and corresponding points of novelty. For additional details and/or possible perspectives of the invention and embodiments, the reader is directed to the Detailed Description section and corresponding figures of the present disclosure as further discussed below.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is a schematic of a drug delivery implant device, according to an embodiment of the present disclosure.

FIG. 1B is a schematic of a drug delivery implant device including an insertion assistance device, according to an embodiment of the present disclosure.

FIG. 2 is a schematic of the ventilation tube inserted through an incision, according to an embodiment of the present disclosure.

FIG. 3 is a schematic of the ventilation tube with the catheter integrated, according to an embodiment of the present disclosure.

FIG. 4A is a graph of day 7 ABR thresholds after sodium thiosulfate (STS) trans-tympanic (TT) injection on the round window membrane for 0.2M 5% STS treated and untreated, and native.

FIG. 4B is a graph of day 7 ABR threshold shifts after STS TT injection on the round window membrane for 0.2M 5% STS treated and untreated.

FIG. 4C is a graph of day 7 ABR thresholds after STS TT injection on the round window membrane for 0.4M 5% STS treated and untreated, and native.

FIG. 4D is a graph of day 7 ABR threshold shifts after STS TT injection on the round window membrane for 0.4M 5% STS treated and untreated.

FIG. 4E is a graph of is a graph of day 7 ABR thresholds after STS intratympanic (IT) injection on the round window membrane for 0.2M 5% STS treated and untreated, and native.

FIG. 4F is a graph of day 7 ABR threshold shifts after STS IT injection on the round window membrane for 0.2M 5% STS treated and untreated.

FIG. 4G is a graph of day 7 ABR thresholds after STS IT injection on the round window membrane for 0.1M 2.5% STS treated and untreated, and native.

FIG. 4H is a graph of day 7 ABR threshold shifts after STS IT injection on the round window membrane for 0.1M 2.5% STS treated and untreated.

FIG. 5 is a schematic showing fluorescence microscopy images of stained cochleae.

FIG. 6A is a graph of hair cell counts loss percent after STS TT treatment.

FIG. 6B is a graph of ABR shifts after STS TT treatment.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

This disclosure is directed towards new drug delivery device designs that circumvent the need for an office procedure by an ear, nose, and throat (ENT) doctor in typical transtympanic injections and enable more frequent dosing regimens. The combination of the device and inner ear medications enables preventative or therapeutic interventions that are difficult or impossible with existing methods. In particular, platinum (II or IV) chelators, such as sodium thiosulfate (STS) can be combined with devices and methods described herein to prevent cisplatin or other platinum anticancer drug induced hearing loss and associated otic disorders.

In general, there can be 2 types of local administration methods: (1) intracochlear injection; and (2) intratympanic injection (also called transtympanic injection).

Some inner ear delivery methods are not repeatable, affordable, and safe. Intracochlear administration includes introducing the drug directly into the cochlea at various sites, including round window membrane, oval window or basal turn of the cochlea. In a clinical setting, direct intracochlear injection is performed only during surgery under general anesthesia. The procedure is highly invasive and carries a potential risk to introduce pathogens in the inner ear when the cochlea is opened via a cochleostomy. It is only used for one time treatment such as gene therapy for serve hearing loss patients. Besides the intracochlear injection, a variety of tools including cochlear implant coating and advanced devices has been employed for intracochlear administration.

Intracochlear injection requires surgical operation, is highly invasive, and is considered for one-time treatment. A physician can obtain access to the cochlea through a surgical procedure and injection medications via a punctured hole on round window membrane or a drilled hole on the bony structure of the cochlea or the oval window. The procedure is highly invasive and required to be performed in a hospital setting. Currently, it is only suitable for a few special occasions, such as co-administration with cochlear implant surgery, one time treatment (e.g., gene therapy), and for deaf patients. It is not suitable for otoprotective applications.

A cochlear implant is a hearing device with an array implanted in the scala tympani that can stimulate directly the spiral ganglions neurons on a damaged cochlea. It is implanted by a surgery, which nevertheless leads to histological damages during insertion. Companion medications can be administrated during the surgery to improve the outcome of the cochlear implant. Furthermore, biodegradable polymeric coatings on the cochlear implant surface represent an interesting strategy to deliver drug along with the implant. The electrode can be embedded within a polymeric matrix containing a drug that will be released into the scala tympani for a prolonged period of time.

One modified intracochlear injection is to use an implanted pump to continuously infuse medications into the inner ear. Reciprocating drug delivery includes delivering soluble drugs to closed fluid spaces in the body such as the cochlea via a single cannula without consequent fluid volume change. The core of the microfluidic reciprocating reservoir is a system for perfusion of drugs into the cochlear perilymph through a single hole in the basal turn of the cochlea. A critical issue for using this device consists in obtaining an overall size consistent with surgical implantation into the mastoid cavity behind the ear. The wearable device is tested in animals, but not in humans yet.

In practice, intratympanic (IT) injection is performed in outpatient clinic. Intratympanic injection is minimally invasive and has been widely used with an established safety record. A physician opens an incision on the tympanic membrane and inserts a blunt syringe needle through the incision and blindly instills medication into the middle ear cavity and fills the middle ear cavity ensuring that the drug is in contact with the round window. The medication reaches the round window area, and the medicine can permeate through the round window membrane to enter the inner ear. The medication can leak out of middle ear cavity through the eustachian tube, and the patient is asked not to swallow any of the medication and must remain relatively still in the supine position with the patient's head turned both during the injection and for some time afterwards (e.g., 15 minutes) to allow the medication to diffuse through the round window. The method has been widely used by physicians to treat inner ear or middle ear disorders, for example IT injection of corticosteroids. Overall, the intratympanic injection is less invasive and lower in cost compared to intracochlear injection. However, it is still a poor solution since it is required to be performed by a professional otologist with the assistance of an endoscope in an ear, nose, and throat (ENT) medical practitioner's office. Furthermore, the frequency of injection is limited to avoid the risk of sustained perforation in tympanic membrane. However, this technique generally requires repeated injections, such as 2-5 times a week because the injected liquid is rapidly eliminated from the middle ear via the Eustachian tube. There is the aforementioned risk of tympanic membrane perforation due to frequent injection. Other adverse reactions related to intratympanic administration are pain or vertigo after injection.

Some studies have been done on these delivery methods in animal and human settings. In animal studies, intratympanic injection of STS prevented the cisplatin induced hearing loss. In contrast, intratympanic administration of STS has been explored in multiple clinical trials, but no meaningful hearing protection has been reported yet. A challenge associated with the intratympanic injection of STS in a clinical setting includes the fact that the clearance rate of STS in the perilymph is rather rapid. Therefore, it is ideal to shorten the time gap between the intratympanic injection of STS and the cisplatin infusion. However, intratympanic injection is performed by an ENT doctor in an ENT office. The cisplatin infusion site is unlikely suitable for the current intratympanic injection. This creates a logistics challenge in coordination of the time for intratympanic injection and cisplatin infusion, which was not a problem in the preclinical animal studies.

In order to reduce the dosing frequency of the IT injection, various hydrogel formulations have been evaluated to extend the contact of medication to the round window membrane. Some typical hydrogels are based on poloxamer 407, hyaluronic acid and chitosan. The clinical significance of hydrogel formulation is still to be determined. There are some potential complications associated with hydrogel formulation, such as transient conductive hearing loss, ototoxicity, etc.

In a clinical trial, the sponsor reported hearing protection when a high concentration hydrogel formulation of STS was administrated via an intratympanic route between 1-3 hours prior to the cisplatin infusion. Besides the logistical challenges, there are the following drawbacks in the STS formulation and method proposed and described by the sponsor.

First, the STS concentration is higher than 0.5M with a calculated osmolarity of approximately 1500 mOsm/L, which is likely irritating and/or potentially damaging to the middle ear mucosal surface. In the reported clinical observation, nearly all patients reported ear pain, and some of the patients also reported tinnitus with a trend of correlation to the dose level (dose concentration).

Second, a viscous hydrogel formulation was used to improve the inner ear exposure of thiosulfate. However, the viscous hydrogel formulation might be associated with lower spreadability and generating bubbles during the injection. Therefore, the contact of the formulation to the very small round window membrane can be compromised and the overall effective exposure of STS in the inner ear might not be better than an aqueous formulation. For example, an injection of the viscous hydrogel formulation can result in air bubbles mixed into the hydrogel formulation that contacts the round window membrane, which can result in the air bubbles (i.e., no formulation) also contacting the round window membrane and lead to a less effective or entirely ineffective delivery of the thiosulfate. Instead, a non-hydrogel formulation, such as an aqueous solution, will advantageously submerge and uniformly contact the round window membrane.

Third, the sponsor also suggested to administer STS minimally 1 hour prior to the cisplatin infusion in order to avoid potential interaction between the STS in circulation and the infused cisplatin. This regimen would waste the high STS concentration in the inner ear within the first hour post STS dosing. The high dose level (0.5M or above) also increased the potential impact on the systemic exposure of thiosulfate. This could potentially impact the intended antitumor activity of cisplatin.

Additionally, the current intratympanic injection is not suitable for frequent repeated dosing. Certain cisplatin dosing regimens require daily infusions. For example, the testicular cancer regimen is to dose cisplatin every day for 5 days every cycle. This would require an STS intratympanic injection every day for those 5 days every cycle. This is not possible due to the high risk associated with the frequent repetitive and prolonged tympanic membrane perforation and the logistical difficulty of scheduling both each day within the timeframe required for both to be effective. Additionally, pre-hydration can be a regimen used prior to the infusion of cisplatin to mitigate or minimize potential renal damage caused by cisplatin. Hydration typically requires 1 hour or more to complete. Therefore, the proposed STS intratympanic delivery and the cisplatin infusion can be further separated apart.

Thus, there is a need for an effective and practical delivery method that can be performed without an otologist and no requirement for an ENT office visit during the injection.

Implantation of a (micro) catheter is difficult, requires surgery to manipulate the tympanic membrane, and requires a hospital operation. The microcatheter has two lumens, one for infusion of drugs and another for fluid withdrawal. The bulbous tip of 1.5, 2.0, or 2.5 mm diameter is placed and compressed in the round window niche. The other end of the catheter exits from the outer ear canal and can be connected to various pumping systems for infusion of drugs such as micropumps and osmotic pumps. The large catheter often leaves a defect in the tympanic membrane. Dislocation of the catheter opening tip and the round window membrane can also occur due to variable interpersonal round window niche sizes. Therefore, this can negatively impact delivery efficiency and consistency. Round window membrane erosion is a risk due to prolonged contact with the implanted structure. General anesthesia is required to expose the round window niche after performing a tympanomeatal flap procedure. Also, two operations are required for this technique-one for catheter insertion and another for its removal, where the associated complications include catheter dislocation, obstruction, and the formation of mild granulation tissue in the middle ear cavity. Regardless, the catheter, micro-pump, surgical procedure, and subsequent hospitalization are very expensive, implantation is difficult, and the large catheter often leaves a defect in the tympanic membrane. For intratympanic injection, it is important to retain an adequate path for air flow. The microcatheter implant has no room left after insertion of catheter into the aperture of tube.

Another technique includes the implantation of a ventilation tube on the tympanic membrane. The patient can self-dispense the drug fluid into the external ear canal whereby it is intended to pass through the opening into the middle ear, and then potentially the inner ear. This has the disadvantages of passage of the liquid drug into the middle ear is inhibited by the surface tension of the liquid and pressing the tragus of the ear multiple times to pump drug into middle ear cavity. The volume that can be introduced into the middle ear cavity is low and unlikely to reach the round window membrane. The loss of the drug fluid through the eustachian tube more readily occurs than the entry of drug fluid through the small hole in the tube. Therefore, the delivery to the inner ear has poor efficiency.

In addition, infectious debris can be carried into the middle ear from the external canal, with the risk of creating a middle ear infection. In practice, it was only used for the delivery of antibiotics to the middle ear cavity to treat middle ear infection.

Inserting a wick between the external ear canal and the middle ear can be performed. The Silverstein MicroWick is an absorbent polyvinyl acetate wick with a length of 9 mm and a diameter of 1 mm. This device is inserted through a ventilation tube in the tympanic membrane and placed overlying the round window. The insertion procedure requires only local anesthesia. Then, the patient instills the drug solution into the external ear canal, usually several times a day and for several weeks. The drug absorbed by the wick is delivered in contact with the round window for a passive diffusion to the inner ear. This method has the disadvantages of challenging microwick placement, possible patient noncompliance, errors in following directions, confusion of edications, failure of some or all of the instilled drops to reach or pass through the wick, delivery variability due to environmental factors (e.g. humidity) and chronic perforations due to the extended use of the wick. The wick, in contact with fluid (e.g. fluid produced due to the incision on tympanic membrane), can expand in size significantly, and make it difficult to insert through the ventilation tube. The capillary effect decreases in the subsequent dose. The contact of the wick to the round window membrane can cause increased fibrosis on the round window membrane and potentially damage hearing.

As the discussion above illustrates, there is a need for an improved method and device for treating inner ear diseases. The method should avoid invasive procedures and hospitalization, and minimize the need of an ENT office visit. It would be advantageous to enable dosing outside the ENT office and by a non-otologist or the patient him/herself. The method can enable repeat dosing with minimal ENT office visits.

An advantage of the device designs discussed herein lies with the unique capability for the patient to administer dosing after implantation without the hazards and medicine loss of other patient dosing methods. The ability to self-dose decreases treatment costs, scheduling logistics, and time commitments while increasing effectiveness. While the focus of the present disclosure is related to hearing loss and other otic disorders caused by platinum anticancer drugs, the advantages of the described devices and methods will also extend to other applications, such as medicine delivery to the inner ear for other medical conditions.

The present disclosure relates to new medical devices designed to repeatedly deliver medicine to the inner ear without medical personnel. The design and application method described herein provides an increase in effectiveness and a decrease in cost to the patient.

Inner ear drug delivery remains quite challenging, and current methods are associated with various disadvantages. The present disclosure addresses this by providing an implant device that allows for a one-time implantation via a trained professional followed by administering or introducing the desired drug into the tympanic cavity using the implant device through a catheter having an inlet with a connector for attaching a fluid injecting device, such as a syringe. Notably, the administering of the drug can be performed by a non-trained professional at any time and in any location, and multiple subsequent injections can also be performed at any time and in any location, and the drug will always be administered successfully to the inner ear since the implant device (the catheter inlet) can be fastened securely to the patient and an outlet tip of the catheter is inserted through the tympanic membrane into the tympanic cavity such that any administered drug, e.g., a thiosulfate solution, fills the tympanic cavity until the drug contacts the round window membrane disposed therein.

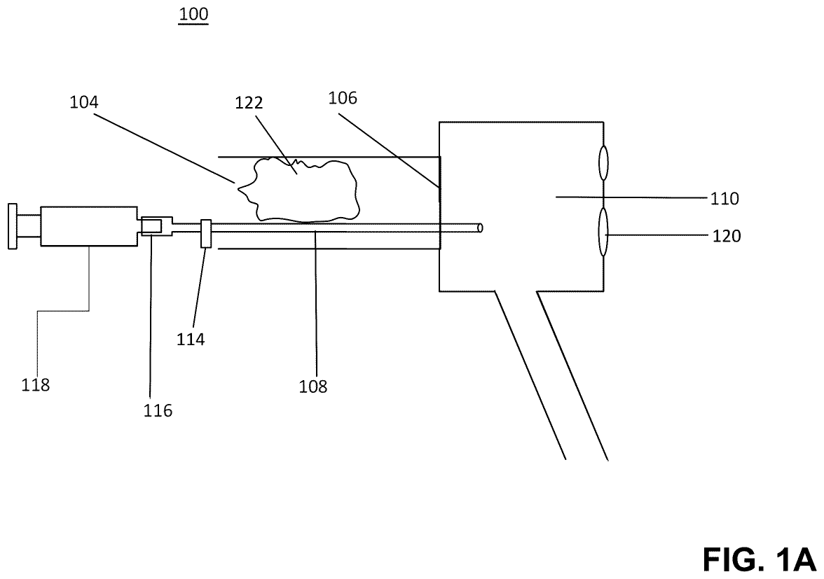

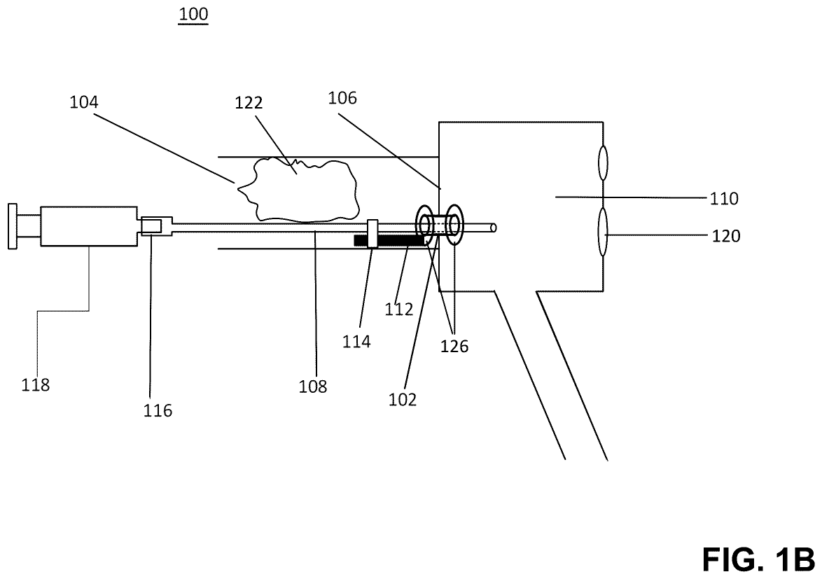

Referring now to the drawings, FIG. 1A is a schematic of a drug delivery implant device 100, according to an embodiment of the present disclosure. In an embodiment, the drug delivery implant device 100 includes a catheter 108 arranged or delivered down an ear canal 104 and inserted into a tympanic membrane 106 (also known as the ear drum). For example, an incision can be made on the tympanic membrane 106 through which the catheter 108 is inserted. The tympanic membrane 106 in a patient is a thin, usually circular piece of tissue that separates an outer ear and the ear canal 104 from a middle ear. Thus, the catheter 108, when inserted into the tympanic membrane 106, can extend from the ear canal 104 side, through the tympanic membrane 106, and into a tympanic cavity 110 side. In an embodiment, an outlet tip of the catheter 108 can be inserted through the tympanic membrane such that the outlet tip extends into the tympanic cavity with a distance of from 2 mm to 5 mm from the tympanic membrane. The tympanic membrane 106 tissue can be stretched and therefore the catheter 108 can be secured to the tympanic membrane 106 via a clamp force exerted on the catheter 108 by the tympanic membrane 106. An inlet end of the catheter 108 can be secured outside of the patient's ear canal 104 via a tape 114. In an embodiment, feature 114 can be a suture or a medical adhesive tape.

FIG. 1B is a schematic of a drug delivery implant device 100 including an insertion assistance device, according to an embodiment of the present disclosure. In an embodiment, a ventilation tube 102 is arranged or delivered down the ear canal 104 and inserted into the tympanic membrane 106. Therefore, the ventilation tube 102, when inserted into the tympanic membrane 106, can have one side disposed on the ear canal 104 side and another side disposed on a tympanic cavity 110 side. In an embodiment, the ventilation tube 102 can be biased to seat centrally along an axis of the ventilation tube 102 on the tympanic membrane 106. The tympanic membrane 106 tissue can be stretched and therefore the ventilation tube 102 can be secured to the tympanic membrane 106 via a clamp force exerted on the ventilation tube 102 by the tympanic membrane 106. As described below, the catheter 108 can be inserted through the ventilation tube 102 in order to pass through the tympanic membrane 106 and into the tympanic cavity 110. Thus, in an embodiment, the ventilation tube 102 is configured to receive the catheter 108.

In an embodiment, to further secure the ventilation tube 102 to the tympanic membrane 106, the ventilation tube 102 can include flanges 126 on a first end and an opposite, second end of the ventilation tube 102. The flanges 126 can be wider in diameter than a center portion of the ventilation tube 102. Therefore, when the ventilation tube 102 is inserted into the tympanic membrane 106, the clamp force of the tympanic membrane 106 adjusts an arrangement of the ventilation tube 102 such that the tympanic membrane 106 clamps onto the narrower center portion of the ventilation tube 102 and the wider flanges 126 at the first end and the second end of the ventilation tube 102 prevent the tympanic membrane 106 from slipping off the ventilation tube 102 at either end. Additionally or alternatively, the ventilation tube 102 includes only one of the flanges 126 at the first end or the second end. The single flange 126 can, for example, prevent excess insertion into the tympanic cavity 110 while not providing an obstacle for insertion on the opposite side. Concomitantly, the opposite side without the flange 126 does not secure the ventilation tube 102 to the tympanic cavity 110 as strongly, but also does not provide additional resistance (and thus introduce potential discomfort to the patient) as the ventilation tube 102 is removed.

In an embodiment, the catheter 108 inserted through the tympanic membrane 106 (either directly or via the ventilation tube 102) has the outlet tip disposed in the tympanic cavity 110 without coming into contact with a wall of the tympanic cavity 110. For example, the catheter 108 does not contact the round window membrane along the wall of the tympanic cavity 110.

In an embodiment, the catheter 108 is arranged or inserted into the tympanic cavity 110 through the ventilation tube 102. The catheter 108 has an outer diameter smaller than an inner diameter of the ventilation tube 102 to allow airflow out of the tympanic cavity 110 even when the catheter 108 is in use. The catheter 108 extends from the tympanic cavity 110 to outside the ear canal 104. The catheter 108 can have position markers near the outlet tip of the catheter 108 to facilitate determination of a depth of the catheter 108 while inserting the catheter 108 into the tympanic cavity 110. In an embodiment, a length of the catheter 108 is more than 0.5 cm, or more than 1 cm, or more than 2 cm, or more than 3 cm as measured from the inlet end to the outlet tip. In an embodiment, the catheter 108 has an outer diameter of from 0.3 mm to 1.3 mm, or 0.3 to 0.7 mm. For example, the catheter 108 is a Neo-Magic 1.9 Fr×6 cm EPIV. For example, the catheter 108 is a Premicath Model 1261.080 central venous polyurethane catheter.

In an embodiment, the catheter 108 can be made from a soft material (e.g., thermosensitive polyurethane, silicon, etc.), and the material can become softer when the catheter 108 is inserted into the tympanic cavity 110 where the temperature is close to that of the body. That is, the catheter 108 tube softens upon warming such as upon insertion in the tympanic cavity 110 and warming to body temperature. The catheters 108 are configured to soften upon warming, but not to such a degree that the catheter 108 tube integrity is compromised. This can minimize the potential of scratching any surface in the tympanic cavity 110 by the catheter 108. For example, one type of catheter can be described as those used with neonatal and premature newborns for vascular introduction of drugs and nutrients.

In an embodiment, the catheter 108 is secured to the tympanic membrane via a clamp force of the tympanic membrane on the catheter 108. In an embodiment, the catheter 108 is secured to the ventilation tube 102 to provide stability, keep the catheter 108 outlet tip from touching a round window membrane 120, decrease the risk of injury from movement of the catheter 108, and maintain adequate spacing to allow pressure balancing during injection. FIG. 1B depicts a tab 112 and tape 114 to fasten the catheter 108 to the tab 112.

With reference to both FIGS. 1A and 1B, an earmold 122 can be arranged in the ear canal 104 proximal to the catheter 108 also disposed in the ear canal 104. The earmold 122 can fill a remaining volume of the ear canal 104 and be configured to prevent motion of the catheter 108. Combined with the tape 114, the earmold 122 can provide additional security or stability of the catheter 108 in the ear canal 104. The earmold 122 can be, for example, a medical sponge, foam pad, or inflatable or fillable membrane. In an embodiment, the earmold 122 can be formed as part of the catheter 108 or attached to the catheter 108 disposed in the ear canal 104. For example, the earmold 122 can be a fillable membrane attached to the catheter 108 and inflated or filled with a fluid upon insertion of the catheter 108 into the ear canal 104.

In an embodiment, the inlet end of the catheter 108 includes a connector 116 which can be fitted with a syringe 118 (or pump or other similar device) for injection of a fluid stored therein. The connector 116 can be a Luer lock. Similarly, the tape 114 can be tape, but additionally or alternatively, the tape 114 is a fastener that uses glue, a suture, sonic welding, etc. The connector 116 includes a cap when not connected to the catheter 108 and in use.

In an embodiment, the syringe 118 is a pump including processing circuitry communicatively coupled to a remote processing device, such as a smart phone, tablet, personal computer, or the like, and configured to transmit data to and receive data from the remote processing device. The pump can include sensors, such as a pressure sensor, and transmit sensor data to the remote processing device. The remote processing device can, based on the sensor device, transmit instructions to the processing circuitry. The instructions can be, for example, to run the pump and inject the fluid at a set injection flow rate. The instructions can be, for example, to stop the pump if a pressure threshold is reached to prevent injury to the patient. The patient can, via the remote processing device, adjust settings, such as the pressure threshold and injection flow rate. The remote processing device can be set to instruct the pump to run an injection schedule or regimen automatically based on the treatment for the patient.

In an embodiment, the initial implantation can be performed by an ENT doctor in a medical office. The doctor can further secure the catheter 108 near an opening of the ear canal 104 to the patient's head using a fastener such as tape, glue, sutures, etc. The administration of medication thereafter (the fluid in the syringe 118 configured to be injected) can be performed anywhere and at any time by a nurse, or by the patient if needed. For example, once a patient has the implant, the administration of cisplatin chelator such as sodium thiosulfate (STS) can be performed right before the initiation of cisplatin infusion without an additional ENT visit or ENT coordination. Additional dosing can be performed as needed.

In an embodiment, as shown in FIG. 1A and FIG. 1B, the drug delivery implant device 100 is easier to implant because the drug delivery implant device 100 has small size and sits freely in the tympanic cavity 110 and the outlet tip of the catheter 108 does not touch the round window membrane. The thermoplastic feature of the catheter tube makes it easy to implant as it is hard at cold temperature, but it softens upon warming up in body temperature. Notably, this includes when the catheter 108 is inserted through the tympanic membrane 106 directly and therefore in contact with the tympanic membrane 106, as well as when the catheter 108 is inserted through the ventilation tube 102 that is inserted into the tympanic membrane 106. Upon injection of the fluid through the catheter 108, the tympanic cavity 110 can fill with the fluid. However, the fluid filling the volume of the tympanic cavity 110 can result in an exerted pressure on walls of the tympanic cavity 110. Without a path for the air in the tympanic cavity 110 to escape as the fluid fills the tympanic cavity 110, the exerted pressure can cause discomfort for the patient and hard for injection. As such, the ventilation tube 102 can include a venting feature or path.

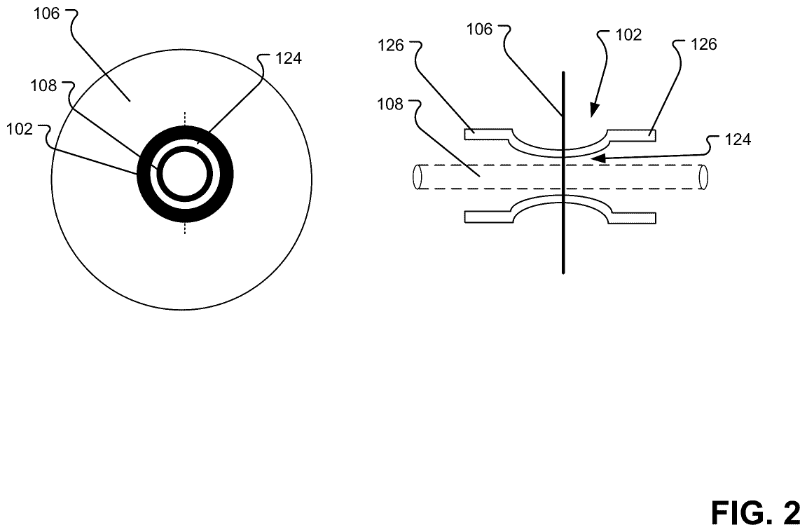

To this end, FIG. 2 is a schematic of the ventilation tube 102 inserted through the incision (dotted line), according to an embodiment of the present disclosure. In an embodiment, the ventilation tube 102 includes a vent 124. In an embodiment, the vent 124 comprises the additional volume between the inner diameter of the ventilation tube 102 and the outer diameter of the catheter 108. The additional volume comprising the vent 124 can therefore never be blocked by the catheter 108 and therefore always provides a passage or path for fluid, such as air, in the tympanic cavity 110 to escape. The vent 124 also provides the additional advantage of allowing the catheter 108 more movement in the vent 124, which can help during arrangement of the outlet tip of the catheter 108 in the tympanic cavity 110. Further, the vent 124 prevents airflow being forced through the incision, which can cause discomfort to the patient.

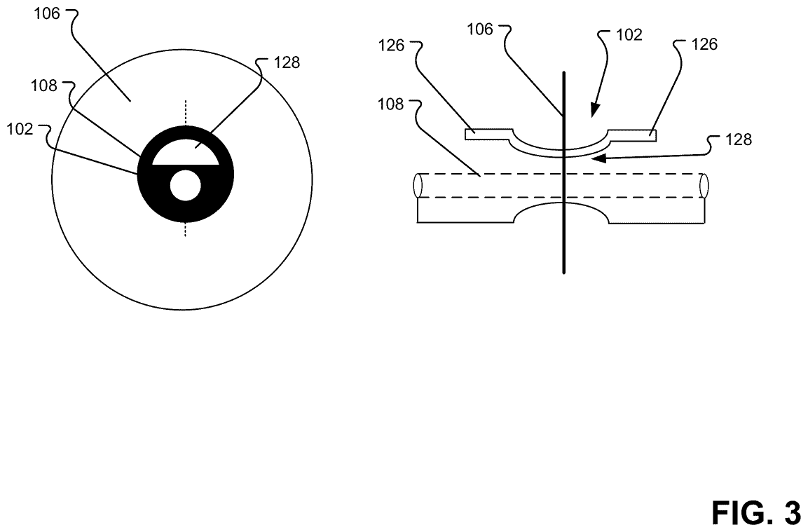

FIG. 3 is a schematic of the ventilation tube 102 with the catheter 108 integrated, according to an embodiment of the present disclosure. In an embodiment, the ventilation tube 102 includes an integrated vent 128. The integrated vent 128 is an opening or aperture that extends from the first end of the ventilation tube 102 through to the second end of the ventilation tube 102. The catheter 108 is also formed as part of the ventilation tube 102 and likewise extends from the first end of the ventilation tube 102 through to the second end of the ventilation tube 102. For example, the catheter 108 is formed along a wall of the ventilation tube 102. Additionally or alternatively, the end of the ventilation tube 102 in the ear canal 104 with the catheter 108 formed as part of the ventilation tube 102 can extend all the way through the ear canal 104 and out of the ear canal 104. Additionally or alternatively, the end of the ventilation tube 102 in the tympanic cavity 110 can be configured to be cut or trimmed to a desired length per patient. The length can be based on a distance between the ventilation tube 102 and the round window membrane, wherein the length is short enough that the outlet tip of the catheter 108 does not contact the round window membrane.

Notably, the ventilation tube 102 with the catheter 108 integrated and the integrated vent 128 can allow the doctor or specialist installing the drug delivery implant device 100 to only perform a single installation into the tympanic membrane 106. A second step to insert the catheter 108 through the ventilation tube 102 is not required. Moreover, since the catheter 108 is integrated in the ventilation tube 102, the catheter 108 does not need to be secured to the ventilation tube 102. Of course, an end of the integrated catheter 108 can still include a feature to connect to additional tubing connected to the syringe 118 (or pump, etc.) Again, since the integrated vent 128 is formed as part of the ventilation tube 102, the integrated vent 128 will remain open and unobstructed by the catheter 108, and the integrated vent 128 prevents airflow being forced through the incision, which can cause discomfort to the patient

In an embodiment the catheter can be made of a radiopaque material so that it is visible by x-rays. For example, the catheter material could include barium sulfate.

Some of the medications suitable for use with the delivery implant 100 are summarized in Table 1.

TABLE 1
Medications suitable for use with the delivery implant.

| MoA | Compounds | Indications |
|---|---|---|
| c-Jun N-terminal | AM-111 | ISSNHL |
| kinase inhibitor | | |
| Reduce formation of | Strekin | SSNHL |
| reactive oxygen | | |
| species | | |
| Glucocorticoid | Dexamethasone | Meniere's Disease |
| NMDA receptor | AM-101, OTO-313 | Tinnitus |
| antagonist | (gacyclidine) | |
| Gamma scretase | LY3056480, | SNHL |
| inhibitor | PIPE-505 | |
| Neurotrophin | BDNF, NT-3 | SNHL |
| APAF-1 inhibitor | LPT99 | Drug induced |
| | | hearing loss |
| Soidum thiosulfate | | Cisplatin induced |
| | | hearing loss |
| Anti-apoptotic/ | AC-102 | SSNHL |
| inflammatory | | |
| Hair cell regeneration | FX-322 | SNHL |
| LXR modulator | AF243 | |
| SGK2 agonist | BN201 | SNHL |
| PGPF2 alpha agonist | Latanoprost | Meniere's Disease |
| PPAR agonist | STR001 | |
| | (pioglitazone) | |

Other medicines that can be used with the device and method of the present disclosure are described in Table 2.

TABLE 2
Indications and corresponding agents for treatment.

| Indications | Agents |
|---|---|
| Meniere's disease | Glucocorticoid, e.g. dexamethasone, Methylprednisolone, |
| | Dexamethasone-phosphate, 6α-Methylprednisolone 21- |
| | hemisuccinate, Methylprednisolone suleptanate |
| Idiopathic sudden | Glucocorticoid, e.g. dexamethasone, Methylprednisolone, |
| sensorineural hearing loss | Dexamethasone-phosphate, 6α-Methylprednisolone 21- |
| (ISSNHL) | hemisuccinate, Methylprednisolone suleptanate |
| Platinum (cisplatin) | alkaline diethyldithiocarbamate salt, amifostine, methionine, |
| induced hearing loss | N-acetylcysteine, cysteine, 2-aminoethanethiol, glutathione |
| | (GSH) or a C1-C6 alkyl ester thereof, lysine, histidine, |
| | arginine, ethylene diamine tetraacetic acid, dimercaprol, |
| | dimercaptosuccinic acid, dimercapto-propane sulfonate salt, |
| | penicillamine, α-lipoic acid, or fursultiamine |
| Sensorineural hearing loss | neurotrophins, e.g. NT-3, BDNF; Glucocorticoid |
| (SNHL) | |
| Noise induced hearing | neurotrophins, e.g. NT-3, BDNF; Glucocorticoid |
| loss | |
| Tinnitus | c-Jun N-terminal kinase inhibitor (e.g. AM-111); NMDA |
| | receptor antagonist (e.g. AM-101, OTO-313/gacyclidine) |
| hair cell regeneration, | Gamma scretase inhibitor (e.g. LY3056480, PIPE-505); FX- |
| noise induced hearing | 322 |
| loss, SNHL | |

As described herein, an aqueous solution of sodium thiosulfate ranging from 0.1M to 0.5M with a calculated osmolarity of, for example, <1500 mOsm/L, or 300-1500 mOsm/L, was used. That is, the molarity of STS can be, for example, 0.1M, 0.15M, 0.2M, 0.25M, 0.3M, 0.35M, 0.4M, 0.45M, etc. The simple aqueous formulation performs equally or better than a hydrogel. The medication is injected into middle ear cavity via trans-tympanic or intratympanic injection no earlier than 1 hour prior to the start of cisplatin infusion and no later than 8 hours after the completion of cisplatin infusion. The systemic thiosulfate concentration was increased after STS local administration. A dosing volume of 0.1-0.5 mL was used. The Tmax was approximately 30 min and the concentration recovered to normal after around 1 hour. Systemic exposure (Cmax) resulting from lower STS concentration (<0.5M, >0.1M) should not impact cisplatin. Notably, any methods to mitigate platinum induced hearing loss have used compositions greater than 0.5M and/or 1500 mOsm/L. The STS formulation used herein has lower STS concentration (lower osmolarity) and is better tolerated; overly hypertonic formulations are detrimental to the contacted cells and have negative safety consequences. Thus, an upper limit of 600 mOsm/kg has been used.

In an embodiment, a method of administering, to a tympanic cavity, a pharmaceutical composition using a drug delivery device implanted through a tympanic membrane is described. First, the doctor fluidly connects the drug delivery device to an injection system via an inlet of a flexible catheter of the drug delivery device. Then, a volume of the pharmaceutical composition can be introduced, via the drug delivery device, to the tympanic cavity.

In an embodiment, the pharmaceutical composition comprises an effective amount of an aqueous solution of a platinum chelator. The effective amount can be based on, for example, a volume sufficient to fill the tympanic cavity and for the pharmaceutical composition to contact the round window membrane in the tympanic cavity. For example, a volume of the administered pharmaceutical composition is from 0.05 mL to 1.0 mL, or 0.075 mL to 0.75 mL, or 0.1 mL to 0.5 mL.

In an embodiment, the method can further include forming an incision in the tympanic membrane having a length greater than an outer diameter of the flexible catheter and inserting the flexible catheter 108 through the incision. In an embodiment, the flexible catheter 108 when inserted into the tympanic cavity 110 extends into the tympanic cavity 110 but does not contact the round window membrane.

In an embodiment, the platinum chelator solution is a thiosulfate solution having a concentration of from 0.05M to 0.4M, or 0.075M to 0.3M, or 0.1M to 0.25M.

In an embodiment, the thiosulfate solution has a pH of from, for example, 5.0 to 10.0, or 6.0 to 9.75, or 7.0 to 9.5 buffered with boric acid.

In an embodiment, the platinum chelator is a thiosulfate salt.

In an embodiment, the introducing the pharmaceutical composition to the tympanic cavity occurs at from 1 hour prior to 1 hour after administering one or more platinum-based antineoplastic agents, or at from 30 minutes prior to 30 minutes after administering one or more platinum-based antineoplastic agents, or at from 15 minutes prior to 15 minutes after administering one or more platinum-based antineoplastic agents, or at from 5 minutes prior to 5 minutes after administering one or more platinum-based antineoplastic agents.

In an embodiment, the one or more platinum-based antineoplastic agent is cisplatin. In an embodiment, the composition further comprises PD-1 or PDL-1 inhibitors.

In an embodiment, the pharmaceutical composition is administered multiple times per day.

In an embodiment, the pharmaceutical composition is administered at least once per day for at least two days. The treatment period can be, for example, every day, or every other day, or every 3 days, or every n days.

In an embodiment, the flexible catheter has an outer diameter of from 1 Fr to 2 Fr.

In an embodiment, the flexible catheter is constructed of a biocompatible material having a reduced rigidity when exposed to a body temperature of a patient. In an embodiment, the biocompatible material is polyurethane.

In an embodiment, the inlet of the flexible catheter is connected to a connector of the injection system having a Luer lock, the inlet having a complementary lock to the Luer lock configured to twistably receive the connector.

In an embodiment, the method further includes attaching the inlet of the flexible catheter, via a suture, to a surface exterior to an ear canal.

In an embodiment, the method further includes attaching the inlet of the flexible catheter, via an adhesive tape, to a surface exterior to an ear canal.

In an embodiment, the method further includes inserting an outlet of the flexible catheter through the tympanic membrane such that the outlet extends into the tympanic cavity with a distance of from 2 mm to 5 mm from the tympanic membrane.

In an embodiment, the flexible catheter is used on multiple days to administer the platinum chelating agent.

In an embodiment, the platinum chelating agent is at least one selected from the group consisting of an alkali metal thiosulfate salt, an alkaline earth thiosulfate salt, an ammonium thiosulfate salt, and an organoammonium thiosulfate salt. In an embodiment, the platinum chelator is sodium thiosulfate.

In an embodiment, the effective amount of the aqueous solution of the platinum chelator is introduced to a level at or above a round window membrane in the tympanic cavity.

In an embodiment, a method of treating hearing loss associated with a treatment with one or more platinum-based antineoplasic agents is described. First, the doctor delivers the catheter 108 down through the ear canal 104 and inserts the catheter 108 through the tympanic membrane 106 into the tympanic cavity 110. The catheter 108 is inserted through an opening on the tympanic membrane. In an embodiment, the opening is formed via an incision by the doctor. In an embodiment, the opening is formed via the ventilation tube 102 also delivered down through the ear canal 104 and inserted into the tympanic membrane 106. The doctor secures the inlet end of the catheter 108 to the patient's outer ear. The subsequent injection of a pharmaceutical composition comprising an aqueous solution of a platinum chelator can be performed by the doctor, other trained profession, or even the patient him/herself.

In an embodiment, the platinum chelator solution is administered to the patient by intratympanic or transtympanic injection via the catheter 108. For example, the platinum chelator solution is stored in the syringe 118 attached to the inlet end of the catheter 108.

In an embodiment, the platinum chelator solution is administered to fill the tympanic cavity 110 and cover the round window membrane disposed therein. This can be, for example, a volume of from 0.1 mL to 1.0 mL, or 0.15 mL to 0.6 mL, or 0.2 to 0.35 mL, or 0.1 mL to 0.5 mL.

In an embodiment, the intratympanic or transtympanic injection via the catheter 108 occurs at a set timeframe prior to beginning a cisplatin infusion. For example, the set timeframe can be from 0 to 1 hour prior, or 0 to 30 minutes prior, or 0 to 5 minutes prior. Advantageously, since the injection can occur without the ENT doctor, the patient can perform the injection him/herself before the cisplatin infusion at the location where the cisplatin infusion occurs.

In an embodiment, a subsequent intratympanic or transtympanic injection, or a booster dose, of the platinum chelator solution can be administered. In an embodiment, the subsequent intratympanic or transtympanic injection via the catheter 108 occurs at a set timeframe after the prior intratympanic or transtympanic injection. For example, the set timeframe can be from 1 hour to 12 hours after, or 1 hour to 6 hours after, or 1 hour to 3 hours after the prior intratympanic or transtympanic injection.

In an embodiment, the platinum chelator is a thiosulfate.

In an embodiment, thiosulfate refers to salts of thiosulfuric acid, e.g., sodium thiosulfate Na₂S₂O₃. In an embodiment, thiosulfate refers to esters of thiosulfuric acid, e.g., O,S-dimethyl thiosulfate CH₃—O—S(═O)₂—S—CH₃. In an embodiment, the thiosulfate is a thiosulfate salt. Examples of thiosulfate salts include, but are not limited to, lithium thiosulfate, sodium thiosulfate, magnesium thiosulfate, calcium thiosulfate, and potassium thiosulfate. In an embodiment, the platinum chelating agent is at least one selected from the group consisting of an alkali metal thiosulfate salt, an alkaline earth thiosulfate salt, an ammonium thiosulfate salt, an organoammonium thiosulfate salt, and an ester of thiosulfuric acid.

In an embodiment, the thiosulfate is present in the pharmaceutical composition at a concentration of from 0.1M to 0.4M, or 0.125M to 0.3M, or 0.15M to 0.25M.

In an embodiment, the pharmaceutical composition has a pH between 5.0 and 9.5.

In an embodiment, the pharmaceutical composition further comprises a buffer agent.

In an embodiment, the buffer agent is boric acid.

### EXPERIMENTAL RESULTS

### Protocol—Animal Preparation and Methods

Albino guinea pigs (Hartley), body weight at 220-350 g were used in the study. 3 days acclimation was performed. 30 guinea pigs were assigned to PK study. 26 guinea pigs were assigned for pharmacology efficacy study. Animals were excluded in the efficacy study with otitis media after otoscopy and Auditory Brainstem Response (ABR) exam.

### Example 1: the Preparation of the STS Formulation

Formulation 1 (5%, w/v, 0.2M)

1.153 mL of pure water was added to sodium thiosulfate pentahydrate (57.67 mg) in a vial. The resulting mixture was gently vortexed for 1 min and sonicated for 1 min. The solution is ready for use freshly.

Formulation 2 (10%, w/v, 0.4M)

1.324 mL of pure water was added to sodium thiosulfate pentahydrate (132.39 mg) in a vial. The resulting mixture was gently vortexed for 1 min and sonicated for 1 min. The solution is ready for use freshly.

Formulation 3 (2.5%, w/v, 0.1M)

2.424 mL of pure water was added to sodium thiosulfate pentahydrate (60.60 mg) in a vial. The resulting mixture was gently vortexed for 1 min and sonicated for 1 min. The solution is ready for use freshly.

### Example 2: Pharmacology Efficacy Study

### Step 1: Preparation of Cisplatin Dosing Formulation

10 mL of saline (0.9% w/v sodium chloride in water) was added to cisplatin dry powder (20 mg) in a vial. The resulting mixture was gently vortexed for 1 min and sonicated for 1 min. The solution is ready for use (2 mg/mL) freshly.

### Step 2: ABR Measurement

The 26 animals were anesthetized with zolazepam hydrochloride (Zoletil) and Xylazine and were recorded for their auditory brainstem responses (ABR) using TDT RZ6 Multi-I/O processor. Acoustic stimuli were delivered via an earphone. Needle electrodes were placed near the ear canal at the caudoventral position, the vertex of the skull, and a ground at the lower leg. The stimulus level was from 10 to 90 dB in 5 dB steps, and the tone-pip frequencies were 4, 24, and 32 kHz. The ceiling sound pressure level was 90 dB. ABR threshold was observed by visual inspection of stacked waveforms as the lowest sound pressure level, at which the waveform was above the noise floor.

Prior to the cisplatin infusion, ABR baseline data from 26 animals were recorded bilaterally from each animal. Elevated ABR data related to otitis media or genetic problems were excluded as the baseline.

Animals were measured again 1 week after STS local delivery and cisplatin infusion treatment. A comparison was performed between the treated ear and the untreated contralateral ear and the baseline.

### Step 3: Dosing Procedure

### Sodium Thiosulfate Trans-Tympanic (TT) Injection

The subjection guinea pig was anesthetized with zolazepam hydrochloride (Zoletil) and Xylazine. A solution of freshly prepared sodium thiosulfate solution was loaded in a Hamilton syringe with 26 G needle. The needle was carefully inserted into the left middle ear cavity via the tympanic membrane. ˜50 μL of dosing solution was injected. The animal was positioned for ˜30 min prior to waking up for the cisplatin infusion.

### Sodium Thiosulfate Intra-Tympanic (IT) Injection

After the guinea pig is anesthetized with the cocktail (Zoletil and Xylazine), it is put in prone neutral position. A postauricular incision is made to expose bulla. Using a 22-gauge sharp needle, a hole is made on the bulla by means of surgical microscope. Visualize the round window through the hole which is facing upward. Carefully drop 10 uL freshly prepared STS on the round window niche without overflowing to the other place of the middle ear. Keep it in neutral position for 30 minutes while closing the wound with suture. ˜30 minutes, the animal was applied cisplatin intravenously.

### Cisplatin Infusion

Cisplatin saline solution (10 mg/kg) was infused through a foot dorsal venin (about 1 min infusion) after 30 min post the injection of sodium thiosulfate solution. 7 days after infusion, all animals were tested ABR again. Selecting some cochleae for histology studies.

### Step 4: Efficacy Exams

### ABR Recorded as Below:

All groups (2.5%, 5% and 10% sodium thiosulfate) showed nearly completed protection of the sodium thiosulfate treated ears. Animals showing significant signs of otitis media/middle ear inflammation were excluded from the analysis.

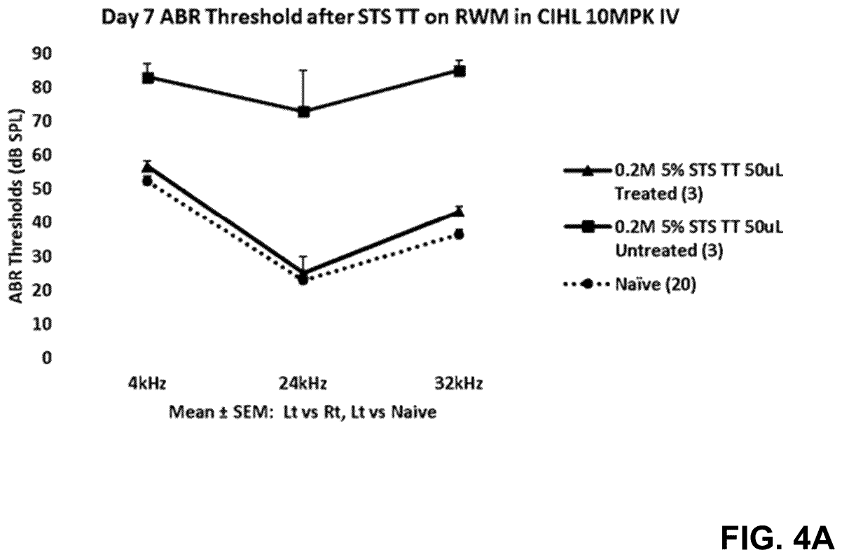

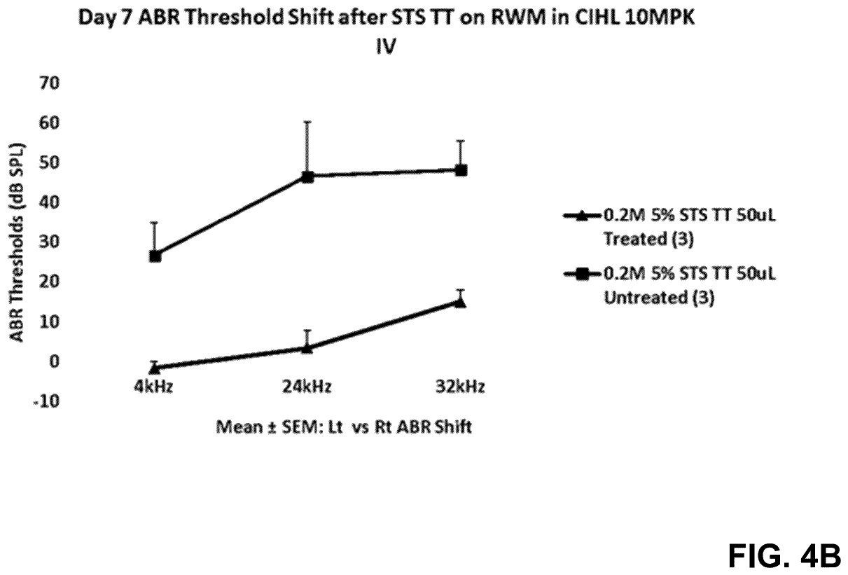

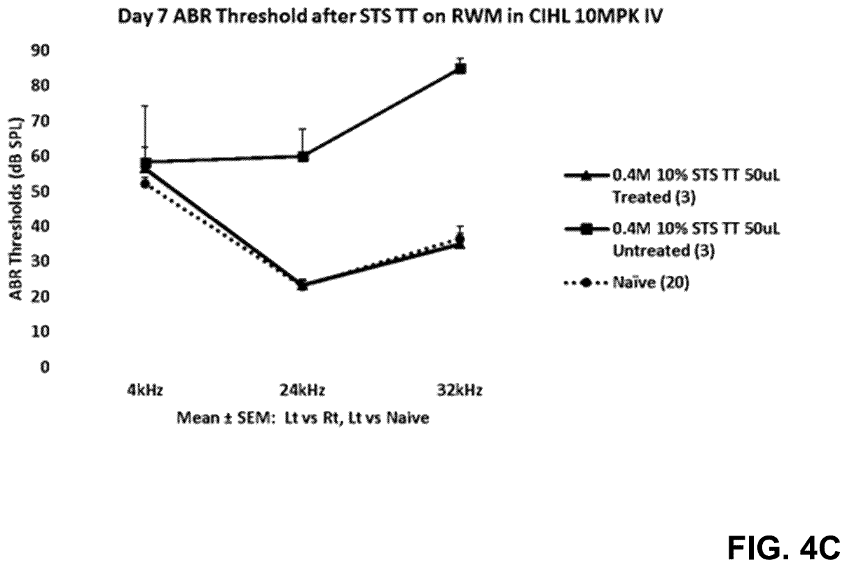

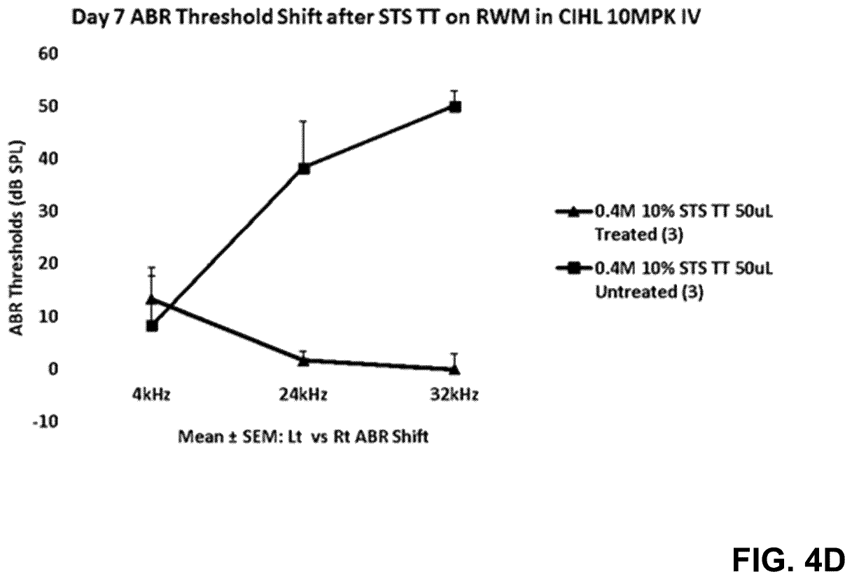

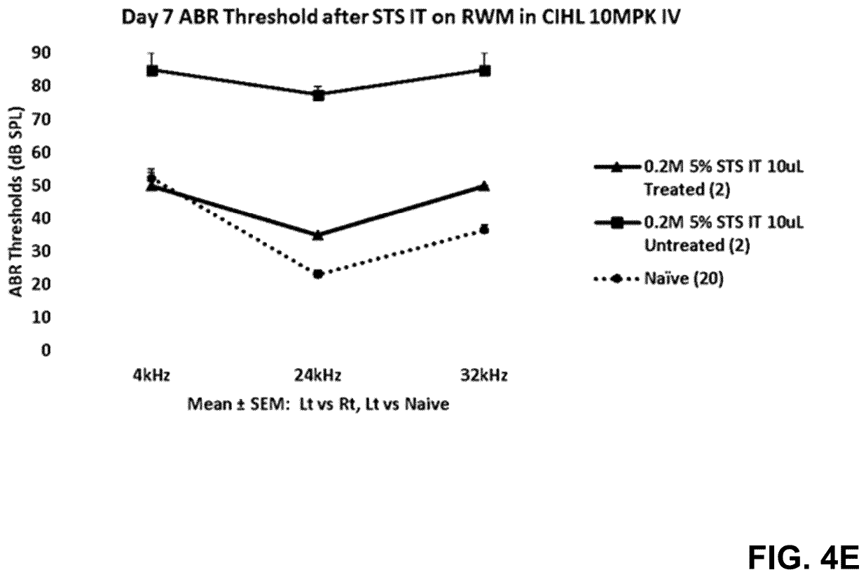

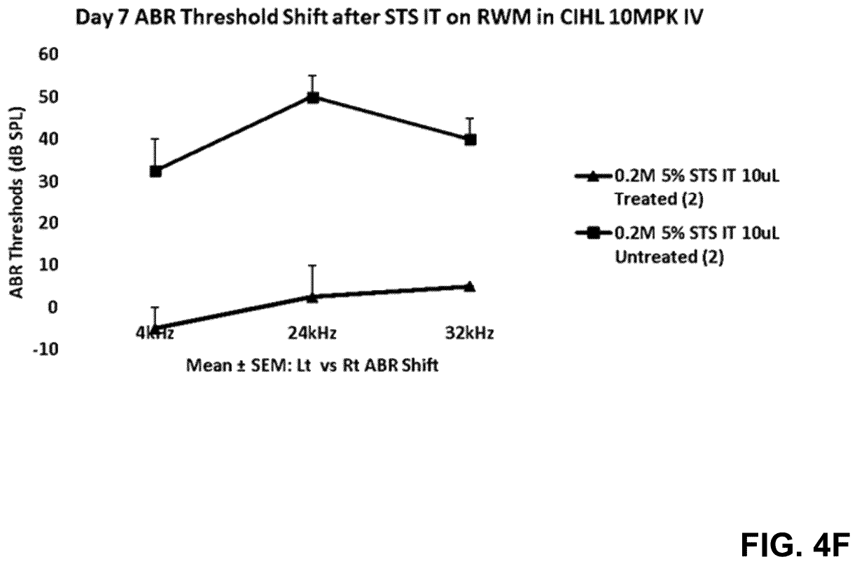

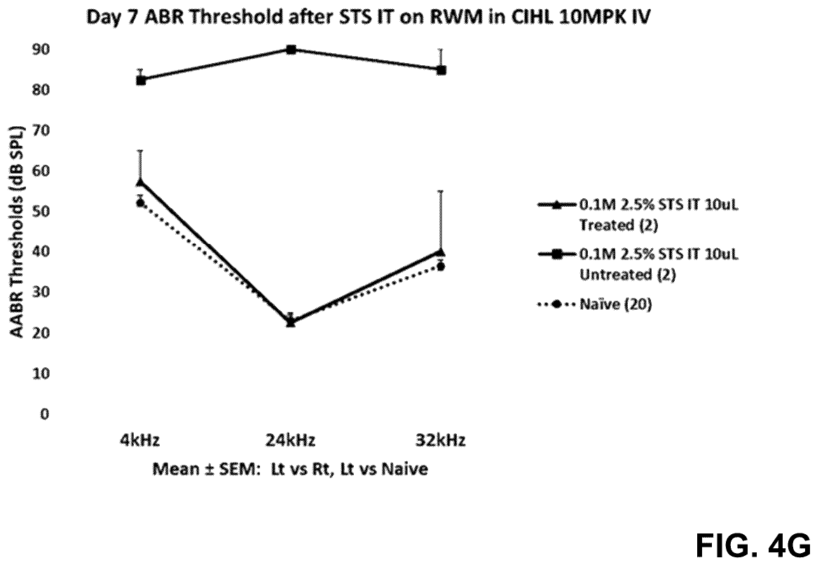

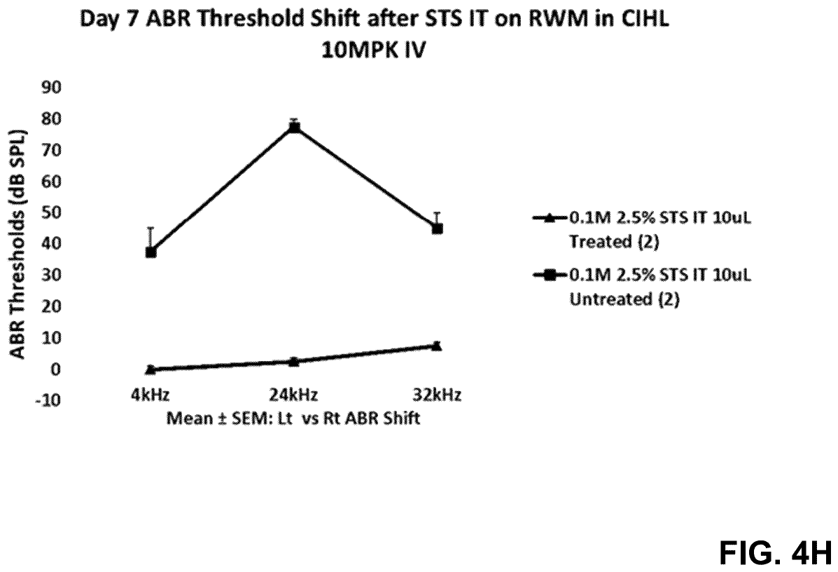

FIG. 4A is a graph of day 7 ABR thresholds after STS TT injection on the round window membrane for 0.2M 5% STS treated and untreated, and native.

FIG. 4B is a graph of day 7 ABR threshold shifts after STS TT injection on the round window membrane for 0.2M 5% STS treated and untreated.

FIG. 4C is a graph of day 7 ABR thresholds after STS TT injection on the round window membrane for 0.4M 5% STS treated and untreated, and native.

FIG. 4D is a graph of day 7 ABR threshold shifts after STS TT injection on the round window membrane for 0.4M 5% STS treated and untreated.

FIG. 4E is a graph of is a graph of day 7 ABR thresholds after STS IT injection on the round window membrane for 0.2M 5% STS treated and untreated, and native.

FIG. 4F is a graph of day 7 ABR threshold shifts after STS IT injection on the round window membrane for 0.2M 5% STS treated and untreated.

FIG. 4G is a graph of day 7 ABR thresholds after STS IT injection on the round window membrane for 0.1M 2.5% STS treated and untreated, and native.

FIG. 4H is a graph of day 7 ABR threshold shifts after STS IT injection on the round window membrane for 0.1M 2.5% STS treated and untreated.

### Cochleae Confocal Microscopy

Extraction of temporal bones from guinea pig skull. Post-fix temporal bones in 10% NBF 16-24 hours. Decalcify the temporal bones 7 days in 10% EDTA. Micro dissect organ of Corti from cochlea for surface preparation. Immunostaining with Fluorescently Tagged Antibodies to dissected basilar membranes. Phalloidin (green) counterstained the structure of the three rows of OHCs and one row of IHCs. Pou4f3 (red) stained the three rows of OHCs and one row of IHCs. DAPI (blue) stained limbus and spiral ligament tissue. Take a photo on a mounted slide by using a fluorescent microscope. Show the location of 4, 8, 16, 24 and 32 kHz on the image. Count inner hair cells (IHC) and outer hair cells (OHC) in the 200 um distance range

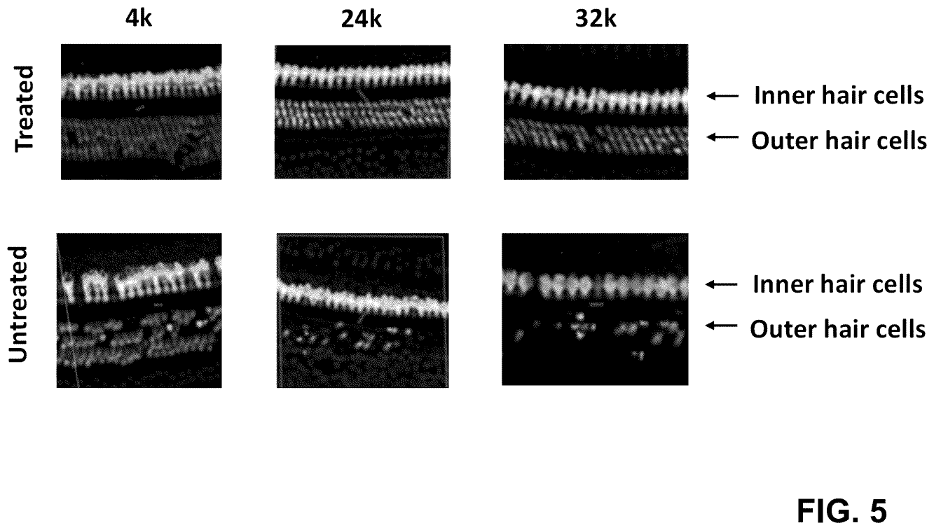

FIG. 5 is a schematic showing fluorescence microscopy images of stained cochleae.

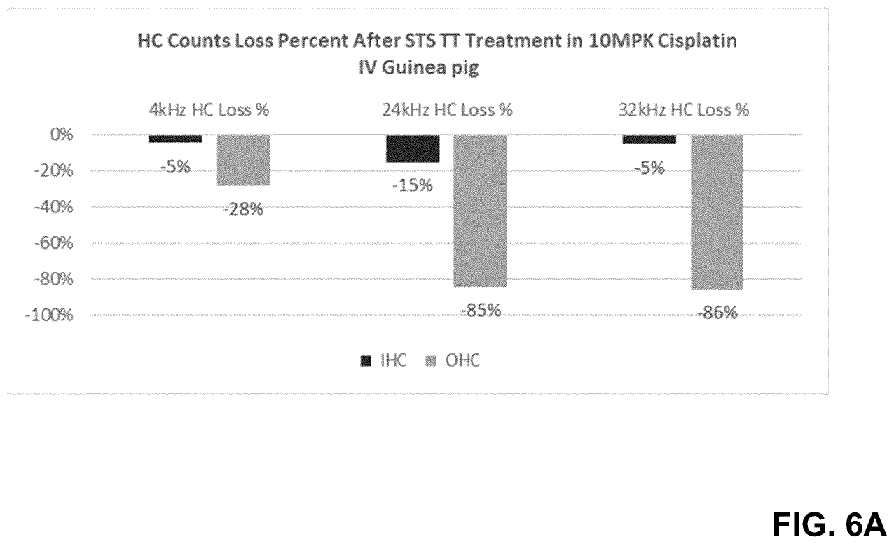

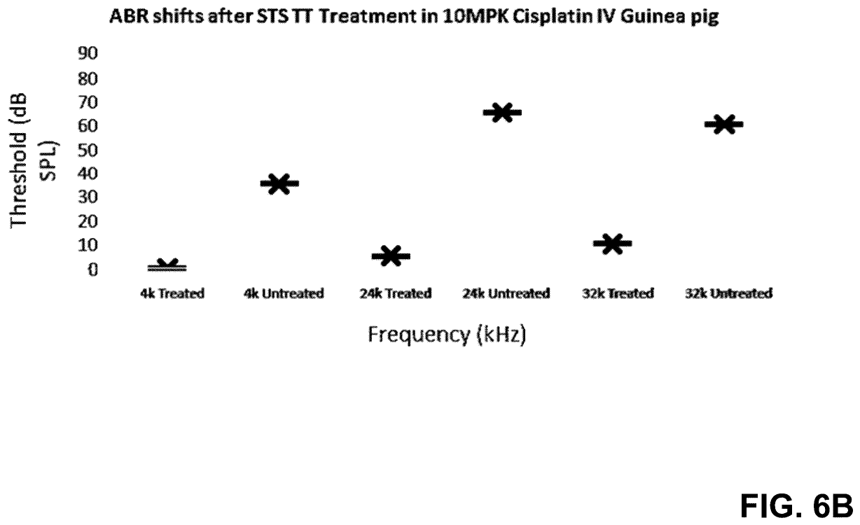

FIG. 6A is a graph of hair cell counts loss percent after STS TT treatment.

FIG. 6B is a graph of ABR shifts after STS TT treatment.

### Example 3: Pharmacokinetic Study

The time-concentration data was used for calculations. The area under the concentration-time curve (AUC) was determined by the trapezoidal rule. Terminal elimination half-lives of sodium thiosulfate (STS) in perilymph and plasma were determined by use of WIN NONLIN version 2.0 SCI.

### Animals' Preparation

30 guinea pigs (250-350 g) without sign of otitis media stayed for ˜3 days acclimation, and ready for PK study. Two anesthesia methods (Zoletil+Xylazine with head up 30 min) and Isoflurane (animal wake up immediately post-injection)] were used. 15 animals for each group.

### Sodium Thiosulfate Trans-Tympanic (TT) Injection

The subjection guinea pig was anesthetized with zolazepam hydrochloride (Zoletil) and Xylazine or isoflurane inhalation. A solution of freshly prepared sodium thiosulfate solution was loaded in a Hamilton syringe with 26 G needle. The needle was carefully inserted into the left middle ear cavity via the tympanic membrane aiming to cochlear round window. ˜50 μL of dosing solution was injected. The animal was positioned head up for ˜30 min prior to waking up in Zoletil and Xylazine or wake up immediately in isoflurane inhalation.

### Perilymph Collection

After euthanasia, the animal was stripped of excess skin and muscle tissue to obtain a complete auditory bulla, and the bulla wall was cut with small forceps to expose the cochlea. The basal turn of bulla was cleaned by using a small cotton ball. The cochlear bottom circle and the round window were coated with bio glue. After drying, a unique micro hole was hand-drilled in the top circle of the cochlea. ˜5 uL volume of perilymph was then collected using a microcapillary inserted into the cochlear top circle. Perilymph samples were added to a vial containing 15 μL of ACN/H2O (v/v, 1:1) stored at −80° C. until analysis.

### Plasma Collection

In C-subgroup of each group, animal blood was systemically drawn (˜100 uL) via bilateral saphenous vein at the multiple time points. Put the collection blood in heparinized tube and stay ˜20 min. ˜50 μL of plasma was collected in microtube and put in −80° C. for future analysis.

### Bio-Analytical Method

STS concentrations were quantitatively analyzed by HPLC using an LC-20AT with a SPD-M20A UV-Vis detector (Shimadzu, Kyoto, Japan) and a LiChrospher RP-select B LiChroCART 250-4 anion exchange column (EMD Millipore Corp, Darmstadt, Germany). All samples were prepared using a method similar to that described by Togawa et al. (1992).

As described above, in vivo efficacy data (via guinea pig trials and cisplatin hearing loss model) was obtained to demonstrate a simple aqueous formulation without a gelling agent can achieve similar or better delivery to the inner ear. Further, full hearing protection was achieved at lower dose levels. For example, the 0.2M concentration achieved said full hearing protection as compared to 0.5M or higher. The lower concentrations still maintained or improved patient comfort levels.

In an embodiment, the pharmaceutical composition comprises an aqueous solution of a platinum chelator at a concentration between 0.1M and 0.5M, and having a calculated osmolarity of about 300 to 1,500 mOsm/L.

In an embodiment, the platinum chelator is sodium thiosulfate.

In an embodiment, the thiosulfate is present in the pharmaceutical composition at a concentration of from 0.1M to 0.4M.

In an embodiment, a salt of thiosulfic acid includes lithium thiosulfate, sodium thiosulfate, magnesium thiosulfate, calcium thiosulfate, and potassium thiosulfate.

In an embodiment, the platinum chelator is alkaline diethyldithiocarbamate salt, amifostine, methionine, N-acetylcysteine, cysteine, 2-aminoethanethiol, glutathione (GSH) or a C1-C6 alkyl ester thereof, dimercaptosuccinic acid, dimercapto-propane sulfonate salt, penicillamine, α-lipoic acid, or fursultiamine.

In an embodiment, the thiosulfate concentration is between about 0.1M and 0.3M.

In an embodiment, the thiosulfate concentration is between about 0.15M and 0.25M.

In an embodiment, the aqueous solution is a water-based solution having a pH between 5 and 9.5.

In an embodiment, the aqueous solution includes a buffer agent.

In an embodiment, the buffer agent is boric acid.

In an embodiment, a method for the prevention of platinum-induced hearing loss in a subject undergoing a treatment with one or more platinum-based antineoplastic agents includes administering the aqueous solution of thiosulfate agent to the subject by intratympanic or transtympanic injection via a catheter, an outlet of the catheter being inserted into a middle ear cavity through an opening on a tympanic membrane at the end of an ear canal, and the aqueous solution of thiosulfate agent filling the middle ear cavity and covering a round window membrane disposed in the middle ear cavity.

In an embodiment, the volume of the injected aqueous solution of thiosulfate agent is 0.1 mL-0.5 mL.

In an embodiment, an inlet of the catheter is arranged outside the outer ear.

In an embodiment, the inlet end of the catheter includes a Luer lock for connecting to a syringe.

In an embodiment, the catheter has outer diameter of from 0.3 mm to 1.0 mm, or 0.3 mm to 0.6 mm, or 1 Fr to 2 Fr.

In an embodiment, a material of the catheter is biocompatible and soft, such as polyurethane and silicon. In an embodiment, biocompatible can be generally defined as compatibility with living tissue or a living system by assuring that the product poses minimal toxicity, injury potential, or physiological/immunological reactivity.

In an embodiment, the opening on the tympanic membrane is an incision.

In an embodiment, the opening on the tympanic membrane is a ventilation tube mounted on the tympanic membrane through an incision on the tympanic membrane.

In an embodiment, the outlet tip of the catheter is inserted through the opening or incision on the tympanic membrane and into the middle ear cavity.

In an embodiment, the outlet tip of the catheter is inserted through an aperture of the ventilation tube and into the middle ear cavity.

In an embodiment, the catheter outer diameter is smaller than the opening inner diameter.

In an embodiment, the catheter and the ventilation tube are connected.

In an embodiment, the inlet end of the catheter is a drug reservoir.

In an embodiment, a material of the ventilation tube is fluoroplastics, polyethylene, or silicone with an inner diameter of 0.6 mm to 1.5 mm.

In an embodiment, the ear canal includes or is filled with a medical sponge, ear mold, or ear plug.

In an embodiment, the inlet end of the catheter is secured outside the ear canal by a suture or medical adhesive tape.

In an embodiment, a length of the catheter is more than 0.5 cm, or more than 1 cm, or more than 2 cm, or more than 3 cm as measured from the inlet end to the outlet tip.

In an embodiment, the material of the catheter is radiopaque (e.g., includes barium) for x-ray confirmation of the position of the catheter.

In an embodiment, the catheter has more than one lumens.

In an embodiment, the intratympanic or transtympanic injection of the aqueous thiosulfate solution is performed one or more times during the same day of the platinum-based antineoplastic agent infusion, and any single intratympanic or transtympanic injection is performed no later than 1 hour prior to the beginning of the platinum-based antineoplastic agent infusion.

In an embodiment, the intratympanic or transtympanic injection is between 0 and 1 hour prior to the beginning of cisplatin infusion.

In an embodiment, the intratympanic or transtympanic injection is between 0 and 30 min prior to the beginning of cisplatin infusion.

In an embodiment, the intratympanic or transtympanic injection is between 0 and about 5 min prior to the beginning of cisplatin infusion.

In an embodiment, a subsequent intratympanic or transtympanic injection (a booster dose) is administered between 1 hour and about 12 hours after the prior intratympanic or transtympanic injection.

In an embodiment, a subsequent intratympanic or transtympanic injection (the booster dose) is administered between 1 hour and about 6 hours after the prior intratympanic or transtympanic injection.

In an embodiment, a subsequent intratympanic or transtympanic injection (the booster dose) is administered between 1 hour and about 3 hours after the prior intratympanic or transtympanic injection.

In an embodiment, a drug delivery implant device includes a ventilation tube including a first end, a second end, and an opening extending from the first end to the second end; and a catheter including an inlet end and an outlet tip configured to be inserted through the ventilation tube via the opening, wherein an outer diameter of the catheter is less than an inner diameter of the opening of the ventilation tube, and the catheter is secured to the ventilation tube allowing for airflow through the opening of the ventilation tube.

Embodiments of the present disclosure may also be as set forth in the following parentheticals.

(1) A method of administering, to a tympanic cavity, a pharmaceutical composition using a drug delivery device implanted through a tympanic membrane, the method including: fluidly connecting the drug delivery device to an injection system via an inlet of a flexible catheter of the drug delivery device; and introducing, to the tympanic cavity, a volume of the pharmaceutical composition via the drug delivery device.

(2) The method of (1), wherein the pharmaceutical composition comprises an effective amount of an aqueous solution of a platinum chelator.

(3) The method of either (1) or (2), further including: forming an incision in the tympanic membrane having a length greater than an outer diameter of the flexible catheter; and inserting the flexible catheter through the incision.

(4) The method of any one of (1) to (3), wherein the chelator is a thiosulfate at a concentration of from 0.1M to 0.4M.

(5) The method of any one of (1) to (4), wherein the thiosulfate has a concentration of from 0.1M to 0.3M.

(6) The method of any one of (1) to (5), wherein the thiosulfate has a concentration of from 0.1M to 0.25M.

(7) The method of any one of (1) to (6), wherein the thiosulfate solution has a pH of from 7.0 to 9.5 buffered with boric acid.

(8) The method of any one of (1) to (7), wherein the platinum chelator is a thiosulfate salt.

(9) The method of any one of (1) to (8), wherein a volume of the administered pharmaceutical composition is from 0.1 mL to 0.5 mL.

(10) The method of any one of (1) to (9), wherein the introducing the pharmaceutical composition to the tympanic cavity occurs at from 1 hour prior to 1 hour after administering one or more platinum-based antineoplastic agents.

(11) The method of any one of (1) to (10), wherein the introducing the pharmaceutical composition to the tympanic cavity occurs at from 30 minutes prior to 30 minutes after administering one or more platinum-based antineoplastic agents.

(12) The method of any one of (1) to (11), wherein the one or more platinum-based antineoplastic agent is cisplatin.

(13) The method of any one of (1) to (12), wherein the composition further comprises PD-1 or PDL-1 inhibitors.

(14) The method of any one of (1) to (13), wherein the pharmaceutical composition is administered multiple times per day.

(15) The method of any one of (1) to (14), wherein the pharmaceutical composition is administered at least once per day for at least two days.

(16) The method of any one of (1) to (15), wherein the flexible catheter has an outer diameter of from 1 Fr to 2 Fr.

(17) The method of any one of (1) to (16), wherein the flexible catheter is constructed of a biocompatible material having a reduced rigidity when exposed to a body temperature of a patient.

(18) The method of any one of (1) to (17), wherein the biocompatible material is polyurethane.

(19) The method of any one of (1) to (18), wherein the inlet of the flexible catheter is connected to a connector of the injection system having a Luer lock, the inlet having a complementary lock to the Luer lock configured to twistably receive the connector.

(20) The method of any one of (1) to (19), further comprising attaching the inlet of the flexible catheter, via a suture, to a surface exterior to an ear canal.

(21) The method of any one of (1) to (20), further comprising attaching the inlet of the flexible catheter, via an adhesive tape, to a surface exterior to an ear canal.

(22) The method of any one of (1) to (21), further comprising inserting an outlet of the flexible catheter through the tympanic membrane such that the outlet extends into the tympanic cavity with a distance of from 2 mm to 5 mm from the tympanic membrane.

(23) The method of any one of (1) to (22), wherein the flexible catheter is used on multiple days to administer the platinum chelating agent.

(24) The method of any one of (1) to (23), wherein the platinum chelating agent is at least one selected from the group consisting of an alkali metal thiosulfate salt, an alkaline earth thiosulfate salt, an ammonium thiosulfate salt, and an organoammonium thiosulfate salt.

(25) The method of any one of (1) to (24), wherein the platinum chelator is sodium thiosulfate.

(26) The method of any one of (1) to (25), wherein the effective amount of the aqueous solution of the platinum chelator is introduced to a level at or above a round window membrane in the tympanic cavity.

(27) A method of treating hearing loss associated with a treatment with one or more platinum-based antineoplastic agents, including: attaching a drug delivery implant device to a tympanic membrane of a subject; and administering to the tympanic cavity an effective amount of a pharmaceutical composition comprising an aqueous solution of a platinum chelator.

(28) The method of (27), wherein the drug delivery implant device includes a catheter having an inlet end and an outlet tip, the outlet tip being inserted through the tympanic membrane.

(29) The method of either (27) or (28), wherein the administering is performed until the pharmaceutical composition fills the tympanic cavity and contacts a round window membrane disposed along the wall of the tympanic cavity opposite the tympanic membrane.

(30) The method of any one of (27) to (29), wherein the outlet tip of the catheter extends into the tympanic cavity, but does not contact a wall of the tympanic cavity.

(31) The method of any one of (27) to (30), wherein the attaching the drug delivery implant device to the tympanic membrane further comprises forming an incision in the tympanic membrane having a length greater than an outer diameter of the catheter; and inserting the catheter through the incision.

(32) The method of any one of (27) to (31), wherein the platinum chelator solution is a thiosulfate solution having a concentration of from 0.1M to 0.5M.

(33) The method of any one of (27) to (32), wherein the platinum chelator solution is a thiosulfate solution having a concentration of from 0.1M to 0.4M.

(34) The method of any one of (27) to (33), wherein the platinum chelator solution is a thiosulfate solution having a concentration of from 0.1M to 0.3M.

(35) The method of any one of (27) to (34), wherein the platinum chelator solution is a thiosulfate solution having a concentration of from 0.15M to 0.25M.

(36) The method of any one of (27) to (35), wherein the platinum chelator is sodium thiosulfate (STS).

(37) The method of any one of (27) to (36), wherein an osmolarity of the pharmaceutical composition is from 300 mOsm/L to 1,500 mOsm/L.

(38) The method of any one of (27) to (37), wherein a volume of the administered pharmaceutical composition is from 0.1 mL to 0.5 mL.

(39) The method of any one of (27) to (38), wherein the administering the pharmaceutical composition to the tympanic cavity occurs at a time from 0 to 1 hour prior to administering treatment with the one or more platinum-based antineoplastic agents.

(40) The method of any one of (27) to (39), wherein the administering the pharmaceutical composition to the tympanic cavity occurs at a time from 0 to 30 minutes prior to administering treatment with the one or more platinum-based antineoplastic agents.

(41) The method of any one of (27) to (40), wherein the administering the pharmaceutical composition to the tympanic cavity occurs at a time from 0 to 5 minutes prior to administering treatment with the one or more platinum-based antineoplastic agents.

(42) The method of any one of (27) to (41), wherein the platinum-based antineoplastic agent is cisplatin.

(43) The method of any one of (27) to (42), further comprising administering to the tympanic cavity a second effective amount of the pharmaceutical composition at a time from 1 hour to 12 hours after the initial administering to the tympanic cavity the effective amount of the pharmaceutical composition.

(44) The method of any one of (27) to (43), further comprising administering to the tympanic cavity a second effective amount of the pharmaceutical composition at a time from 1 hour to 6 hours after the initial administering to the tympanic cavity the effective amount of the pharmaceutical composition.

(45) The method of any one of (27) to (44), further comprising administering to the tympanic cavity a second effective amount of the pharmaceutical composition at a time from 1 hour to 3 hours after the initial administering to the tympanic cavity the effective amount of the pharmaceutical composition.

(46) The method of any one of (27) to (45), wherein the drug delivery implant device includes a ventilation tube including a first end, a second end, and an opening extending from the first end of the ventilation tube disposed proximal to an ear canal of the subject to the second end of the ventilation tube disposed proximal to a tympanic cavity of the subject.

(47) The method of any one of (27) to (46), wherein the ventilation tube includes a first flange disposed at the first end of the ventilation tube and a second flange disposed at the second end of the ventilation tube, the first flange and the second flange having an outer diameter wider than an outer diameter of the ventilation tube along a central portion of the ventilation tube disposed between the first flange and the second flange, and the length of the incision in the tympanic membrane is greater than the outer diameter of the flanges.

(48) The method of any one of (27) to (47), further comprising attaching a pumping device to the inlet end of the catheter, the pumping device configured to pump a fluid through the catheter.

(49) A drug delivery implant device, including a ventilation tube including a first end, a second end, and an opening extending from the first end to the second end; and a catheter including an inlet end and an outlet tip configured to be inserted through the ventilation tube via the opening, wherein an outer diameter of the catheter is less than an inner diameter of the opening of the ventilation tube, and the catheter is secured to the ventilation tube allowing for airflow through the opening of the ventilation tube.

(50) The device of (49), wherein the ventilation tube includes an anchor point for securing the catheter.

(51) The device of either (49) or (50), wherein the anchor point for securing the catheter is an elongated tab formed as part of the ventilation tube that extends outwards from the first end of the ventilation tube.

(52) The device of any one of (49) to (51), wherein the catheter is secured to the ventilation tube via an adhesive tape.

(53) The device of any one of (49) to (52), wherein a material of the catheter is polyurethane or silicone.

(54) The device of any one of (49) to (53), wherein the catheter comprises a radiopaque material.

(55) The device of any one of (49) to (54), wherein the catheter includes a connector at the inlet end.

(56) The device of any one of (49) to (55), wherein the catheter includes position markers proximal to the outlet tip.

(57) The device of any one of (49) to (56), wherein the ventilation tube includes a first flange disposed at the first end of the ventilation tube and a second flange disposed at the second end of the ventilation tube, the first flange and the second flange having an outer diameter wider than an outer diameter of the ventilation tube along a central portion of the ventilation tube disposed between the first flange and the second flange.

(58) The device of any one of (49) to (57), wherein the catheter is formed as part of the ventilation tube such that the catheter is formed along a wall of the opening of the ventilation tube and extends straight through the opening of the ventilation tube along the wall, the ventilation tube and the catheter forming a single piece.

(59) The device of any one of (49) to (58), wherein the catheter extends away from the ventilation tube to form an extended portion, the inlet end of the catheter being disposed at an end of the extended portion.

(60) The device of any one of (49) to (59), wherein the catheter extends a predetermined length away from the ventilation tube, the predetermined length being determined by a distance between the ventilation tube and a round window membrane disposed opposite the ventilation tube in a tympanic cavity.

(61) The device of any one of (49) to (60), further comprising a pumping device fluidly connected to the inlet end of the catheter, the pumping device configured to pump a fluid through the catheter.

(62) The device of any one of (49) to (61), wherein the fluid is a pharmaceutical composition configured to mitigate hearing loss associated with a treatment with one or more platinum-based antineoplastic agents, comprising an aqueous solution of a platinum chelator at a concentration of from 0.1M to 0.5M with an osmolarity of from 300 to 1,500 mOsm/L.

(63) The device of any one of (49) to (62), wherein the ventilation tube is disposed in a tympanic membrane, the catheter is inserted through the first end of the ventilation tube disposed in an ear canal to the second end of the ventilation tube disposed in a tympanic cavity including a round window membrane, the outlet tip of the catheter extending into the tympanic cavity, and the pumping device is configured to pump the platinum chelator solution into the tympanic membrane and fill the tympanic membrane until the platinum chelator solution contacts the round window membrane.

(64) The device of any one of (49) to (63), wherein the platinum-based antineoplastic agents is at least one selected from the group consisting of cisplatin, carboplatin, oxaliplatin, nedaplatin, triplatin, tetranitrate, phenanthriplatin, picoplatin, and satraplatin.

(65) The device of any one of (49) to (64), wherein the platinum chelator is a thiosulfate.

(66) The device of any one of (49) to (65), wherein the thiosulfate is present in the pharmaceutical composition at a concentration of from 0.1M to 0.4M.

(67) The device of any one of (49) to (66), further comprising an earmold attached to and surrounding the catheter, the earmold being a reversibly collapsible and fillable container configured to receive an injected fluid and expand to fill a volume adjacent to the catheter and the earmold, the earmold configured to deflate upon the fluid being withdrawn from the earmold.

(68) The device of any one of (49) to (67), wherein the outer diameter of the catheter is less than 1 mm and the inner diameter of the ventilation tube is greater than 1.1 mm.

Obviously, numerous modifications and variations are possible in light of the above teachings. It is therefore to be understood that within the scope of the appended claims, embodiments of the present disclosure may be practiced otherwise than as specifically described herein.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A method of administering, to a tympanic cavity, a pharmaceutical composition using a drug delivery device implanted through a tympanic membrane, the method comprising:
fluidly connecting the drug delivery device to an injection system via an inlet of a flexible catheter of the drug delivery device; and
introducing, to the tympanic cavity, a volume of the pharmaceutical composition via the drug delivery device.

2. The method of claim 1, wherein the pharmaceutical composition comprises an effective amount of an aqueous solution of a platinum chelator.

3. The method of claim 1, further comprising:
forming an incision in the tympanic membrane having a length greater than an outer diameter of the flexible catheter; and
inserting the flexible catheter through the incision.

4. The method of claim 2, wherein the chelator is a thiosulfate at a concentration of from 0.1M to 0.4M.

5. The method of claim 4, wherein the thiosulfate has a concentration of from 0.1M to 0.3M.

6. The method of claim 5, wherein the thiosulfate has a concentration of from 0.1M to 0.25M.

7. The method of claim 4, wherein the thiosulfate solution has a pH of from 7.0 to 9.5 buffered with boric acid.

8. The method of claim 2, wherein the platinum chelator is a thiosulfate salt.

9. The method of claim 1, wherein a volume of the administered pharmaceutical composition is from 0.1 mL to 0.5 mL.

10. The method of claim 2, wherein the introducing the pharmaceutical composition to the tympanic cavity occurs at from 1 hour prior to 1 hour after administering one or more platinum-based antineoplastic agents.

11. The method of claim 10, wherein the introducing the pharmaceutical composition to the tympanic cavity occurs at from 30 minutes prior to 30 minutes after administering one or more platinum-based antineoplastic agents.

12. The method of claim 11, wherein the one or more platinum-based antineoplastic agent is cisplatin.

13. The method of claim 12, wherein the composition further comprises PD-1 or PDL-1 inhibitors.

14. The method of claim 1, wherein the pharmaceutical composition is administered multiple times per day.

15. The method of claim 1, wherein the pharmaceutical composition is administered at least once per day for at least two days.

16. The method of claim 1, wherein the flexible catheter has an outer diameter of from 1 Fr to 2 Fr.

17. The method of claim 1, wherein the flexible catheter is constructed of a biocompatible material having a reduced rigidity when exposed to a body temperature of a patient.

18. The method of claim 17, wherein the biocompatible material is polyurethane.

19. The method of claim 1, wherein the inlet of the flexible catheter is connected to a connector of the injection system having a Luer lock, the inlet having a complementary lock to the Luer lock configured to twistably receive the connector.

20. The method of claim 1, further comprising attaching the inlet of the flexible catheter, via a suture, to a surface exterior to an ear canal.

21. The method of claim 1, further comprising attaching the inlet of the flexible catheter, via an adhesive tape, to a surface exterior to an ear canal.

22. The method of claim 1, further comprising inserting an outlet of the flexible catheter through the tympanic membrane such that the outlet extends into the tympanic cavity with a distance of from 2 mm to 5 mm from the tympanic membrane.

23. The method of claim 2, wherein the platinum chelator is at least one selected from the group consisting of an alkali metal thiosulfate salt, an alkaline earth thiosulfate salt, an ammonium thiosulfate salt, and an organoammonium thiosulfate salt.

24. The method of claim 2, wherein the platinum chelator is sodium thiosulfate.

25. The method of claim 2, wherein the effective amount of the aqueous solution of the platinum chelator is introduced to a level at or above a round window membrane in the tympanic cavity.
